(19) United States
(12) Patent Application Publication
(10) Pub. No.: US 2016/0121061 A1
(43) Pub. Date: May 5, 2016

(54) DEVICE AND METHOD FOR MEASURING A GAS CONSUMPTION PERIOD IN A HOSPITAL BUILDING

(71) Applicant: AIR LIQUIDE SANTE SERVICES, Paris (FR)

(72) Inventors: Guillaume BOUCHER, Nogent-sur-Marne (FR); Jérôme PAULES, Paris (FR)

(21) Appl. No.: 14/893,165

(22) PCT Filed: May 14, 2014
(86) PCT No.: PCT/FR2014/051116

§ 371 Date: Nov. 23, 2015

(30) Foreign Application Priority Data
May 23, 2013 (FR) 1354628

(51) Int. Cl. A61M 16/00; A61M 16/20; G04F 13/06

(52) U.S. Cl. CPC A61M 16/0003; G04F 13/06; A61M 16/0051; A61M 16/0066; A61M 16/201; A61M 2202/0208; A61M 2205/3365; A61M 2205/502; A61M 2205/8206

(57) ABSTRACT

The invention concerns a device (20) for determining the period of consumption of a gas by a patient comprising an internal gas passage (25) with a gas inlet (21) and a gas outlet (22), adjustment means (23) for adjusting a desired gas consumption period (T_{flxé}), display means (24) for displaying at least the desired gas consumption period (Tfixé), means for determining the gas consumption period (5, 6) designed to measure the total gas consumption period (Tconso) during which gas is flowing in said internal gas passage (25), from the gas inlet (21) to the gas outlet (22), information processing means (9) capable of and designed to compare the total gas consumption period (Tconso) determined by the means for determining the gas consumption period (5, 6) with the desired gas consumption period (Tfixé), and flow interruption means designed to stop any flow of gas in said gas passage when the information processing means determine that the total gas consumption period (Tconso) is greater than or equal to the desired gas consumption period (Tfixé).

### CROSS REFERENCE TO RELATED APPLICATIONS

This application is a 371 of International PCT Application PCT/FR2014/051116 filed May 14, 2014 which claims priority to French Patent Application No. 1354628 filed May 23, 2013, the entire contents of which are incorporated herein by reference.

### BACKGROUND

The invention relates to a device and a method for measuring the duration of a gas consumption period which are suited to use within a hospital building comprising a network of gas pipes comprising gas distribution wall outlets arranged on the walls of the rooms occupied by the patients, typically oxygen and medical air wall outlets.

In hospitals it is necessary to be able to monitor the duration of periods during which gas, particularly air and oxygen, is consumed by the patients, accurately and even in the rooms occupied by the patients, namely at the head of the bed where the gases are distributed via gas supply wall outlets.

This is because it is of overriding importance to be able to ensure that the patients inhale the gas prescribed for them for the prescribed duration, namely without interruption before the end of this duration but without extending beyond this duration.

At the present time, when a patient inhales respiratory gas, such as oxygen in the context of oxygen therapy, delivered by a wall outlet supplied by a gas pipe, the duration of the period for which the gas is inhaled is determined empirically by the care workers or by the patient himself, using a watch or the like.

Once the duration has elapsed, the patient removes his mask and/or the care workers stop the supply of gas to the breathing mask.

It will be readily appreciated that this approach is impractical and may lead to timing errors and therefore to inappropriate administration of the therapeutic gas and/or to losses of gas, particularly if the patient removes his mask but the supply to said mask has not been interrupted.

It is therefore necessary to be able not only to determine the duration of the period for which the patient has inhaled the gas but also to interrupt the supply of gas to the patient when this elapsed duration corresponds to the duration medically prescribed by the doctor.

From that starting point, the problem that arises is that of proposing a device that allows the consumption time to be monitored accurately and the distribution of gas to be interrupted automatically when the patient has received enough gas to fulfill his prescription, particularly when the gas administered, such as oxygen or air, is delivered by a wall outlet situated in the room occupied by said patient within a hospital building.

SUMMARY

The solution is therefore a device for determining, namely for measuring and monitoring, the duration of a period during which a gas, such as medical oxygen or medical air, is consumed by a patient, comprising:
an internal gas passage with one (or more) gas inlet and one (or more) gas outlet, the gas flowing along said passage from the inlet toward the gas outlet,
regulating means for setting a desired gas consumption period duration, referred to as T_{fixed},
display means for displaying at least the desired gas consumption period duration T_{fixed},
gas consumption period duration determining means designed to measure the total gas consumption period duration, referred to as T_{consum} for which gas circulates along said gas passage, from the gas inlet toward the gas outlet,
data processing means able and designed to compare the total gas consumption period duration T_{consum} determined by the gas consumption period duration determining means with the desired gas consumption period duration T_{fixed}, and
flow interrupting means designed to interrupt any flow of gas along said gas passage when the data processing means determine that the total gas consumption period duration T_{consum} is greater than or equal to the desired gas consumption period duration T_{fixed}, namely when T_{consum}≧T_{fixed}.

According to circumstance, the device of the invention may comprise one or more of the following technical features:
the gas consumption period duration determining means comprise:
   a rotary member able and designed to be rotated by the flow of gas passing along the gas passage, and
   a Hall-effect sensor collaborating with the rotary member to keep count of the duration of the period for which the rotary member is being rotated by the flow of gas passing along said gas passage.
the regulating means comprise one or several regulating members, particularly one or more keys, buttons, sliders, pressure sensors or presence sensors.
the display means comprise a screen or indicator lamps, for example a liquid crystal screen.
the gas consumption period duration determining means comprise an electronic controller.
the flow interrupting means comprise a valve or gate, preferably controlled by the data processing means, particularly a solenoid-operated valve.
it comprises connection means allowing the device to be fluidically connected to a gas distribution outlet.
it further comprises a power source powering at least the display means and the processing means, the power source preferably comprising one or more electric batteries or cells.
the rotary member is an impeller or a rotor.
the rotary member is able and designed to be turned by a flow of gas, typically air or oxygen, of between 0.1 and 20 I/min, preferably between 0.5 and 15 I/min.
the rotary member comprising at least one magnet that allows the Hall-effect sensor to keep count of the number of revolutions over a given period.

The invention also relates to a method for measuring the duration of the period for which gas is consumed within a hospital building comprising a network of gas pipes comprising gas distribution wall outlets, in which method the duration of the period for which gas is consumed by at least one of said gas distribution wall outlets is measured using a device according to the invention.

For preference, the network of gas pipes comprises:
one or more oxygen pipes carrying gaseous oxygen between an oxygen source and one or more oxygen distribution wall outlets; and/or
one or more air pipes carrying air between an air source and one or more air distribution wall outlets.

Advantageously, the gas distribution wall outlets are arranged in one or more rooms of the hospital building. lo Furthermore, the source of air and/or the source of oxygen are air or oxygen storage tanks respectively or a PSA or VSA unit producing air or oxygen from atmospheric air.

### BRIEF DESCRIPTION OF THE DRAWINGS

The present invention will now be described in greater detail with reference to the attached figures among which:

FIG. 1 schematically indicates the principle of operation of the gas consumption period duration determining means with which the device of FIG. 3 is equipped,

FIG. 2 schematically indicates the interior of the device of FIG. 1 and

FIG. 3 schematically indicates the principle of operation of one embodiment of a gas consumption period duration measurement device according to the invention.

### DESCRIPTION OF PREFERRED EMBODIMENTS

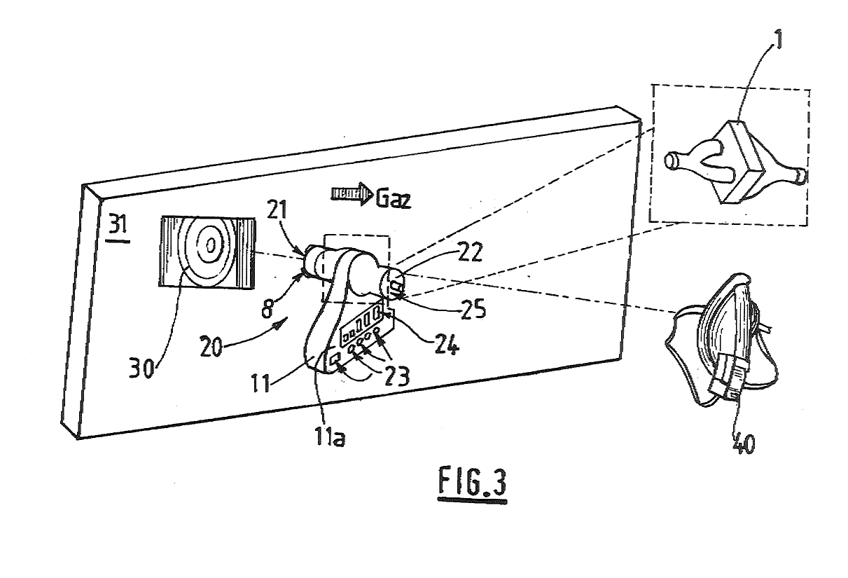

FIG. 3 schematically indicates the principle of operation of one embodiment of a device 20 for measuring the duration of the period for which a gas is consumed by a patient according to the invention, the gas particularly being a gas such as medical oxygen or medical air which are gases customarily conveyed into the treatment rooms or the rooms occupied by the patients in a hospital, via networks of gas pipes and which are distributed thereto by wall outlets 30 worn by the walls 31 before being administered to the patients via interfaces such as breathing masks 40.

This device 20 for determining the duration of the consumption period comprises a casing 11 through which there passes an internal gas passage 25 with a gas inlet orifice 21 and a gas outlet orifice 22, the gas flowing in the direction of the “Gas” arrow, namely in the direction from the gas inlet 21 toward the gas outlet 22.

At the gas inlet 21, the casing 11 comprises connecting means 8, for example a screw thread, a poke yoke system of male/female or similar type allowing the device to be fluidically connected to the gas distribution outlet 30.

Also provided are regulating means used to set a desired gas consumption period duration T_{fixed}, namely to indicate to the device the length of time for which it is necessary to allow gas to circulate along the internal passage 25 so as to supply the patient with respiratory gas as prescribed by the doctor or similar.

The regulating means 23 comprise one or more regulating members such as keys, buttons, numerical keypad, sliders or the like.

The desired gas consumption period duration T_{fixed} is preferably set by the care workers, for example a doctor or a nurse.

Display means 24, such as a screen, allow desired gas consumption period duration T_{fixed} or any other desired information or data to be displayed. For preference, the screen 24 is situated on the front face 11a of the casing 11, namely the face 11a situated on the side of the outlet orifice 22.

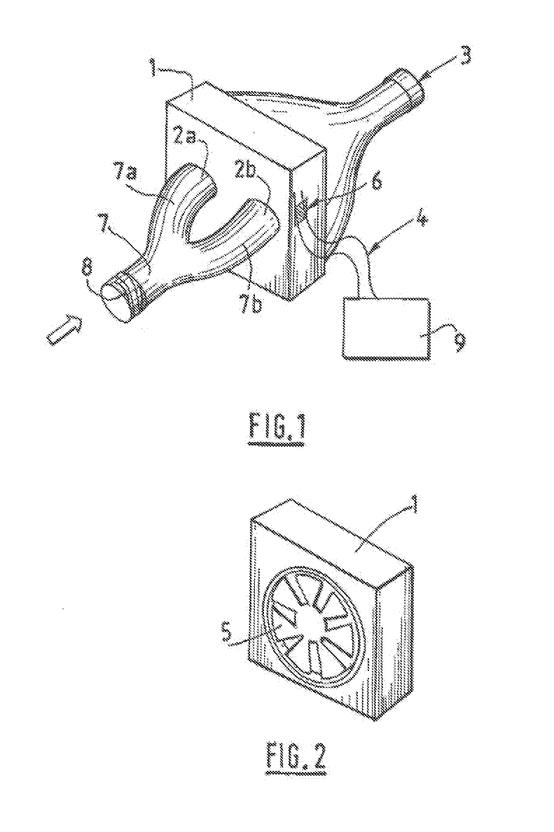

Moreover, the casing 11 also comprises gas consumption period duration determining means 1 designed to measure the total duration T_{consum} of the period for which gas is consumed, namely the total time for which gas circulates along the gas passage from the gas inlet 21 toward the gas outlet 22, before being sent via a flexible hose or the like to the interface 40 connected to the patient. One embodiment of gas consumption period duration determining means 1 is detailed hereinafter and illustrated in FIG. 1 and 2.

Also provided within the casing 1 are data processing means able and designed to compare the total gas consumption period duration (T_{consum}) determined by the gas consumption period duration determining means 1 with the desired gas consumption period duration T_{fixed}. The data processing means are for example an electronic board organized around a programmable microcontroller, or an EE remote transmission of the WiFi/Zigbee type of the consumption to a central console.

Finally, the device 20 of the invention also comprises flow interrupting means, such as a solenoid-operated valve, which are designed to interrupt any circulation of gas along the internal gas passage 25 when the data processing means determine that the total gas consumption period duration (T_{consum}) is greater than or equal to the desired gas consumption period duration (T_{fixed}).

FIG. 1 schematically indicates the principle of operation of the gas consumption period duration determining means with which the device of FIG. 3 is equipped.

Said gas consumption period duration determining means comprise a compartment or chamber 1 with one or more gas inlets 2 and a gas outlet 3, and in which compartment or chamber 1 is arranged a rotary member 5, such as an impeller, as explained hereinafter.

Here, the compartment 1 comprises two gas inlets 2a, 2b each supplied by a single supply pipe 7. This single supply pipe 7 branches into two sub-branches 7a, 7b each of which supplies one of said inlets 2a, 2b of the compartment 1. The benefit of having several inlets 2a, 2b is that the impeller 5 can be set in motion even when the gas flow rates are low, namely typically upwards of around 0.5 l/min, because the gas flow is split over several blades of the impeller 5.

The gas passes through the compartment or chamber 1 containing the impeller 5 and then re-emerges therefrom via an outlet pipe 17 bearing the gas outlet 3. The single supply pipe 7 and the outlet pipe 17 are each connected to and/or form a portion of the internal gas passage 25 passing through the casing 11 of the device 20 of the invention.

Typically, the means for measuring the speed of the gas flow comprise a rotary member 5, such as an impeller or rotor, able and designed to be set in rotation by the gas flow when the latter passes through the compartment 1 in the direction (see arrow in FIG. 1) leading from the inlets 2 toward the outlet 3.

The rotary member 5 collaborates with a Hall-effect sensor 6 so as to determine the duration and/or the speed of rotation of the rotary member 5 by counting the number of revolutions effected by the rotary member 5 over a given length of time, namely over the measurement time.

The duration of the period of rotation of the rotary member 5 in fact corresponds to the duration of the period for which the gas passes through the device 20 and therefore the duration of the period for which the therapeutic gas is administered to the patient, namely the total gas consumption period duration T_{consum}.

The measurements taken by the rotary member 5 are then transmitted, via electrical connections 4, to data processing means 9 such as an electronic controller contained in the casing 11 of the device 20 of the invention so as to determine the total gas consumption period duration T_{consum} from measurements of the rotational speed (i.e. number of revolutions over a given time) of the rotary member 5, and compare this total gas consumption period duration T_{consum} with the desired gas consumption period duration T_{fixed} so as to interrupt any circulation of gas along the internal gas passage 25 when T_{consum}≧T_{fixed}.

Interruption of any circulation of gas along the internal gas passage 25 is achieved by operation of the solenoid-operated valve arranged on the internal passage 25, said solenoid-operated valve being controlled by the data processing means, notably the electronic controller 9.

The electronic controller 9, the display 24 and the solenoid-operated valve arranged on the internal gas passage 25 are powered with electrical current by a battery, a cell or the mains.

Data may then be transmitted by the electronic controller 9 or some other means of transmission, over wires or wirelessly, for example a Wifi, Zigbee, Bluetooth or some other form of transmission to an information system that stores and/or processes the received data, for example a computer, a server or the like.

Alternatively, recording means, for example a memory card, allow the measured data or data calculated from the measurements to be stored.

The device of the invention is particularly well suited to monitoring the gas consumption of the wall outlets arranged in the rooms of a hospital or the like, particularly the consumption of medical air or medical oxygen, within a hospital building, typically at the head of the bed.

1.-12. (canceled)

13. A device (20) for determining the duration of a period during which a gas is consumed by a patient, comprising:
an internal gas passage (25) with a gas inlet (21) and a gas outlet (22),
a regulator (23) for setting a desired gas consumption period duration (T_{fixed}),
a display (24) for displaying at least the desired gas consumption period duration (T_{fixed}),
a gas consumption period duration determining component (5, 6) designed to measure the total gas consumption period duration (T_{consum}) for which gas circulates along said internal gas passage (25), from the gas inlet (21) toward the gas outlet (22),
a data processor (9) able and designed to compare the total gas consumption period duration (T_{consum}) determined by the gas consumption period duration determining component (5, 6) with the desired gas consumption period duration (T_{fixed}), and
a flow interrupter designed to interrupt any flow of gas along said gas passage when the data processor determine that the total gas consumption period duration (T_{consum}) is greater than or equal to the desired gas consumption period duration (T_{fixed}).

14. The device as claimed in claim 1, characterized in that the gas consumption period duration determining component (5, 6) comprise:
a rotary member (5) able and designed to be rotated by the flow of gas passing along the internal gas passage (25), and
a Hall-effect sensor (6) collaborating with the rotary member (5) to keep count of the duration of the period for which the rotary member (5) is being rotated by the flow of gas passing along said internal gas passage (25).

15. The device of claim 1 wherein the regulator (23) comprises one or several regulating members.

16. The device of claim 1 wherein the display (24) comprises a screen and/or an indicator lamp(s).

17. The device of claim 1 wherein the gas consumption period duration determining component comprises an electronic controller (9).

18. The device of claim 1 wherein the flow interrupter comprises a solenoid valve.

19. The device of claim 1 wherein the device comprises a connection (8) allowing the device to be fluidically connected to a gas distribution outlet (30).

20. The device of claim 1 wherein the device further comprises a power source powering at least the display and the processor, the power source comprising one or more electric batteries or power cells.

21. The device of claim 1 wherein the rotary member (5) is an impeller or a rotor, the rotary member (5) comprising at least one magnet.

22. A method for measuring the duration of the period for which gas is consumed within a hospital building comprising a network of gas pipes comprising gas distribution wall outlets, the method comprising the step of measuring the duration of the period for which gas is consumed by at least one of said gas distribution wall outlets using the device of claim 1.

23. The method as claimed in claim 10, wherein the network of gas pipes comprises:
one or more oxygen pipes carrying gaseous oxygen between an oxygen source and one or more oxygen distribution wall outlets; and/or
one or more air pipes carrying air between an air source and one or more air distribution wall outlets.

24. The method as claimed in claim 10, wherein the gas distribution wall outlets are arranged in one or more rooms of the hospital building.
